(19) United States
(12) Patent Application Publication
(10) Pub. No.: US 2005/0186172 A1
(43) Pub. Date: Aug. 25, 2005

(54) Cosmetic composition capable of fighting against skin aging

(75) Inventor: Olivier Courtin, Boulogne-Billancourt (FR)

Correspondence Address: IP GROUP OF DLA PIPER RUDNICK GRAY CARY US LLP, 1650 MARKET ST, SUITE 4900, PHILADELPHIA, PA, 19103, US

(73) Assignee: Laboratoires Clarins, a corporation of France, Neuilly-sur-Seine (FR)

(21) Appl. No.: 11/029,988

(22) Filed: Jan. 5, 2005

Related U.S. Application Data
(63) Continuation of application No. PCT/FR03/02124, filed on Jul. 7, 2003.

(30) Foreign Application Priority Data
Jul. 8, 2002 (FR) 02/08564

(51) Int. Cl. A61K 35/78; A61K 7/06

(52) U.S. Cl. 424/74; 424/757; 424/771

(57) ABSTRACT

A cosmetic composition including an extract of Diospyros kaki, an extract of Pueraria lobata, and one or more formulation agents or additives compatible with cosmetic and dermatological use.

### RELATED APPLICATION

This is a continuation of International Application No. PCT/FR2003/002124, with an international filing date of Jul. 7, 2003 (WO 2004/004680, published Jan. 15, 2004), which is based on French Patent Application No. 02/08564, filed Jul. 8, 2002.

### FIELD OF THE INVENTION

This invention pertains to a cosmetic composition capable of fighting against skin aging containing two plant extracts: Diospyros kaki and Pueraria lobata.

### BACKGROUND

The term “cosmetic composition capable of fighting against skin aging” is understood to mean any cosmetic composition capable of preventing or limiting the consequences of chronological and/or actinic aging at the level of the cutaneous collagen and, consequently, to prevent or limit the cutaneous appearance of visible signs of aging on the skin surface: wrinkles and lines.

Skin aging is characterized among other factors by a global decrease in the thickness of the skin (loss of 6% every ten years starting at birth), a decrease in cell renewal and a decrease in collagen at the level of the dermis associated with an increase in the glycation of the cutaneous proteins.

For women, there is added to this chronological process an aging which can be characterized as hormonal aging subsequent to the drop in the production of estrogens at menopause. This deficiency in estrogens affects the fibroblasts at the dermal level and, consequently, all of the components of the extracellular matrix, collagen in particular.

Nonenzymatic glycation of proteins represents the first cause of modification of cutaneous collagen. Different types of collagen exist and represent 70 to 80% of the dry weight of the dermis. These collagens are constituted of a characteristic triple helix and are organized in fibers. Enzyme-dependent natural couplings normally stabilize the collagen fibrils. However, the glycation phenomenon itself can be responsible for another type of coupling due to a nonenzymatic fixation of the glucose. The collagen fibers then become more rigid, more resistant to pressure and more resistant to collagenases. Renewal of the collagen no longer takes place, the tissues are altered and they become rigid. This becomes an issue of a true process of reticulation with the creation of abnormal and irreversible cross-links. This reticulation is one of the major expressions of skin aging.

The glycated collagens coupled in this manner lose their biological functionality. The tissues become rigid and sclerotic. Glycation is dependent on the possibilities of contact between the circulating glucose molecules and the free amine groups existing either at the N-terminal ends of polypeptide chains or on the lateral lysine chains.

The biochemical mechanism of this reaction is well known and comprises two phases:

1. An early phase (initiation): reaction of reducing sugars (glucose, fructose) with the terminal or lateral amine functional groups of the constitutive proteins of the tissues to produce compounds called “Schiff bases.” These compounds are subsequently stabilized by Amadori rearrangement into ketoamine.

2. A late phase (propagation and termination): the ketoamine functional groups are oxidized into deoxysone in the presence of oxygen and reaction with other basic amino acids such as arginine and lysine belonging to the other proteins (albumin, lipoproteins, immunoglobulins). The result is the formation of complexes with final couplings of the type of pentosidine rings or 6,3-furoyl-1,4-furanyl-imidazole.

One of the important consequences of the glycation of proteins is the creation of free radicals. In effect, when proteins are glycated, they react with oxygen and cause the formation of superoxide type radical groups. These radical groups are capable of initiating the degradation of proteins, altering membrane structures and, finally, disorganizing the extracellular matrix as well as all of its components.

The cutaneous collagen, as well as all of the other components of the extracellular matrix, is also affected by the decrease in estrogens during menopause. In fact, the drop in estrogen levels causes a decrease in the synthesis of collagen by the fibroblasts as well as an increase in its degradation. This collapse occurs quickly and can reach 30% in the first five years of menopause. The production of finer type III fibers contributes to the annual loss of 1% of dermal thickness. Finally, there is also a deterioration in the structural glycoproteins which has the consequence of disturbing the three-dimensional organization of the extracellular matrix.

### SUMMARY OF THE INVENTION

This invention relates to a cosmetic composition including an extract of Diospyros kaki, an extract of Pueraria lobata, and one or more formulation agents or additives compatible with cosmetic and dermatological use.

This invention also relates to a method of preventing skin aging including applying a therapeutically effective amount of the cosmetic composition to skin of a mammal.

This invention further relates to a method of reducing skin aging including applying a therapeutically effective amount of the cosmetic composition to skin of a mammal.

### DETAILED DESCRIPTION

A cosmetic composition useful for fighting against skin aging must therefore contain active ingredients capable of restraining the glycation of proteins and compensating for the estrogen deficit. In this manner, the collagen is protected, collagen being the major molecule of the skin and confers on the skin its mechanical properties. The appearance of wrinkles and the decrease in the cutaneous tonicity are the reflection of the aging of the dermis and notably the aging of its mechanical properties (quantitative diminishment of the collagen network as well as its rigidification).

We have discovered that the combination of two plant extracts, Diospyros kaki and Pueraria lobata, has a protective action on the cutaneous collagen, respectively, an anti-glycation activity and a hormone-like activity, which make these two extracts a useful composition for fighting against skin aging.

The invention pertains most particularly to a composition containing an extract of Diospyros kaki and an extract of Pueraria lobata.

Diospyros kaki, also called Japanese or Oriental persimmon, is a tree originating from hot regions, principally Indonesia. It can be grown in the same regions where olive and fig trees thrive. After the deciduous leaves fall, there can be seen yellow fruit that subsequently turn orange. Local patterns of use have demonstrated the properties of different parts of the tree: the bark of the stems is astringent; the fruit is used in traditional medicine for its antitussive, laxative and nutritive properties; the fresh fruit juice is used against hypertension.

The cosmetic composition of the invention contains an extract of Diospyros kaki. This extract is advantageously obtained from the calyxes of Diospyros kaki. The extract is preferably water soluble and, more particularly, is a hydroglycolic extract. This extract is advantageously obtained according to the following protocol:
(i) the Diospyros kaki calyxes are dissolved in butylene glycol,
(ii) the soluble and insoluble phases are separating by filtration, and
(iii) sterilizing filtration.

The water-soluble and more particularly hydroglycolic extract of Diospyros kaki used in the composition according to the invention has a clear liquid appearance, an amber color and a characteristic odor. It has the following analytical characteristics:
dry matter=9-16 g/l;
total polyphenols=2-4 g/l (flavanol, hydroxycinnamic acid, protocatechuic acid and abscissic acid);
pH=5-6/.

Pueraria lobata, also called kudzu, is a woody vine or liana of the legume family, originating from southern and tropical Asia. This plant bears very fragrant flowers flowing in long clusters and producing brown pods containing 3 to 10 seeds with thick integuments. Its roots are thick or tuberous, its alternating leaves are covered by a very dense brown down. Pueraria lobata contains natural molecules which have an activity similar to that of estrogens. These nonsteroidal substances are isoflavones such as genisteine, daidzeine, coumestrol, pratensein and the like.

The cosmetic composition of the invention contains an extract of Pueraria lobata, advantageously obtained from the roots of the plant. This extract is preferably water soluble and, more particularly, hydroglycolic. This extract is advantageously obtained according to the following protocol:
(a) grinding to 2 mm, 1 weight unit,
(b) addition of ethanol, 5 volume units,
(c) extraction at reflux for 4 hours,
(d) filtration-concentration-evaporation to dryness then addition of glycerin-water, and
(e) filtration at 0.2 μm.

The water soluble more particularly hydroglycolic extract of Pueraria lobata used in the composition according to the invention has a clear liquid appearance of an orangish yellow color and a characteristic odor. It has the following analytical characteristics:
Refraction index (at 25° C.)=1.385-1.405;
Density (at 20° C.)=1.0-1.2;
Isoflavone content (HPLC)=0.3-0.6%;
pH=4.5-6.5.

The composition according to the invention contains:
about 0.1 to about 10% by weight, preferably about 0.5 to about 5% by weight, of Diospyros kaki extract and
about 0.1 to about 10% by weight, preferably about 0.5 to about 5% by weight, of Pueraria lobata extract.

The composition of the invention can also contain any plant extract rich in flavonoids and/or tannins. In fact, the flavonoids interact with the collagen fibers and very effectively protect them from the proteolytic degradation present upon skin aging. Moreover, the rich content of tannins of the plant extracts confer stimulating, antioxidant and protective activities. An antioxidant activity is particularly useful for fighting against skin aging. An extract of wild teasel (Dipsacus sylvestris) is a nonexhaustive example of a tannin-rich plant extract that can be used in the invention. Wild teasel, also called wolf's comb and Venus' basin, is a biannual herbaceous plant of 60 to 150 cm that it is found everywhere in Europe along roads and ditches.

The extract of wild teasel is, more particularly, obtained by digestion in a hydroglycolic mixture from the finely ground aerial parts of the plant. It is then decolored, clarified, standardized and stored.

The plant extracts rich in flavonoids that can be used are advantageously selected from the group including an extract of rosemary (Rosmarinus officinalis), an extract of galangal (Alpina officinarum), an extract of buddleia (Buddleia davidii) and the like.

An extract of rosemary (Rosmarinus officinalis) is advantageously a water-soluble extract obtained from the leaves of this common plant of the Mediterranean region. This extract contains flavonoids, ursolic acid and rosmarinic acid. The extract is obtained according to the method described in FR 9815394.

Galangal (Alpina officinarum) from the ginger family is a shrub originating from East Asia. Its black, gnarled rhizome resembles that of ginger. The galangal extract is a water-soluble extraction of galangal rhizomes obtained by controlled extraction by a water/propylene glycol mixture followed by a centrifugation/filtration step. This galangal extract is rich in phytosterols and flavonoids (1.5 g/l rutin equivalent) such as galangin and kaemferol and in essential oils such as bomeol, cineol, caryophylene and geraniol.

The extract of buddleia (Buddleia davidii) is advantageously a water-soluble extract of buddleia obtained from the dried plants and, more particularly, from the dried flowers and leaves. The dried flowers and leaves are ground, then an ethanol/water extraction is performed. The resultant extract is concentrated after a filtration step and, optionally, a formulation step in glycerin. The water-soluble extract of buddleia that can be used in the composition of the invention contains iridoids, flavonoids (antioxidant agents), esters of caffeic acid (antioxidant agents) and triterpenoids.

The compositions according to the invention can also comprise one or more formulation agents, carriers or additives of known and conventional use in cosmetic and dermatological compositions such as, in the form of nonlimitative examples, softeners, coloring agents, film-forming agents, surface-active agents, perfumes, preservatives, emulsifiers, oils, glycols, vitamins such as vitamin B, UV filters, etc. Based on a knowledge of cosmetics, one skilled in the art can determine which formulation agents to add to the compositions of the invention and in what amounts in relation to the desired properties.

It should be noted that the addition of UV filters to the cosmetic composition enables fighting against the actinic aging induced by UV radiation. The UV filters that can be used in the cosmetic composition can be chemical or mineral filters. The UV filters are advantageously the filters described in FR 2811224.

The compositions can be presented in any form known in the art of cosmetology and dermatology without other pharmaceutical restriction than that of application on the face and on the body. The compositions are advantageously presented in the form of a gel, lotion, cream, emulsion, milk, spray, or the like.

The invention also pertains to the use of a composition for preventing skin aging and/or for fighting against it.

The invention also pertains to the use of extracts of Diospyros kaki and of Pueraria lobata for the preparation of a cosmetic or dermatological composition for preventing skin aging and/or fighting against it.

Other advantages and characteristics of the invention will become apparent from the examples below which pertain to the effect of the extract of Diospyros kaki and of Pueraria lobata on the cutaneous collagen and the examples of formulation which are presented for illustrative purposes and should not be interpreted as limiting the scope of the invention as defined in the appended claims.

I. Effect of the Extract of Diospyros kaki on the Glycation of Proteins

The effect of the extract of Diospyros kaki was studied in vitro on a model based on the measurement of the formation of the derivatives of the glycation reaction between lysine and glucose 6-phosphate. Certain of these derivatives (AGE) are fluorescent. The reaction was measured after 15 days of incubation.

Operating Mode:

A reference molecule was tested in parallel: aminoguanidine.

Test System:

The test system is the reaction mixture containing bovine albumin (0.5 g/ml) and glucose (500 mM) in a phosphate buffer (0.2 M, pH=7.4.).

Incubation Products+Test System:

The products were mixed with the test system in sterile tubes; the tubes were covered with aluminum foil (the reaction must take place shielded from light).

For the experimental conditions in which certain reagents or products are not present, the volume of the QSP tube should be completed with MilliQ water.

After having sealed the openings of the tubes with parafilm (the reaction must take place without oxygen), the different reaction media were placed in a dry oven at 37° C. for 8 days.

Evaluation of the Effects:

After 8 days of incubation, 100 μl was collected from each tube and transferred to a 96-well black plate.

The fluorescence was then read using FLUOstar (BMG) (excitation at 355 nm, emission at 460 nm). The results are expressed in arbitrary fluorescence units.

The relative fluorescence for the “product+bovine albumin” (=interference) is to be subtracted from the “product+bovine albumin+glucose” data.

Results:

After 8 days of incubation of bovine albumin with an overcharge of glucose, the formation of glycation cross-linking was seen. It was evaluated by taking advantage of the fluorescence properties of the bovine albumin-glucose adduct.

The kaki extract present in the test system prevented the cross-linking between the bovine albumin and the glucose in a quite effective manner (Table 1).

TABLE 1
Anti-glycation activity of the Diospyrus kaki extract.

| | | Interference | Intensity | |
|---|---|---|---|---|
| | Intensity of | (without | of real | % |
| | fluorescence | glucose) | fluorescence | inhibition |
| | (arbitrary | (arbitrary | (arbitrary | of the |
| Product | units) | units) | units) | glycation |
| Control | 50,229.5 | 291 | 49,938.5 | / |
| (BSA + glucose) | | | | |
| Aminoguanidine | 13,011.5 | 17,537 | 0 | 100 |
| (10 mM) | | | | |
| Diospyros kaki | 34,131.5 | 21,093.5 | 13,038 | 73.9 |
| 0.5% | | | | |
| Diospyros kaki | 34,282 | 24,883 | 9,399 | 81.2 |
| 1% | | | | |
| Diospyros kaki | 9,869 | 33,893 | 5,976 | 88.0 |
| 3% | | | | |
| Diospyros kaki | 43,052.5 | 37,550.5 | 5,502 | 89.0 |
| 5% | | | | |

II. Effect of the Extract of Pueraria lobata on the Synthesis of Proteins

The effect of the extract of Pueraria lobata was studied in vitro on fibroblast cultures: measurement of the stimulation of the cell metabolism and the synthesis of proteins.

Principle:

Normal human fibroblasts were cultured in a conventional DMEM medium supplemented with fetal calf serum in the presence of Pueraria lobata extract. After an optimal period of incubation, the total quantity of intracellular and extracellular proteins was determined according to the BCA method. The cell metabolism was quantified by the MTT method which indicates the level of mitochondrial respiration.

Results:

The Pueraria lobata extract stimulated the metabolism and synthesis of proteins in a dose-dependent manner.

The MTT values (mitochondrial cellular respiration) and quantity of proteins per 1000 cells were determined in parallel but independently on the same cell cultures. The Pueraria lobata extract at 0.3% augmented the MTT conversion by 44%. This increase in the MTT conversion reached 66% for the 1% extract of Pueraria lobata. For the same time, the total quantity of proteins synthesized by the cells reached 99%.

III. Examples of Formulations According to the Invention

Cream

| | |
|---|---|
| Emulsifiers | 7.000 |
| Triglycerides | 10.000 |
| Shea butter | 2.000 |
| Silicon oils | 1.500 |
| Neutralized gelling agent | 0.600 |
| Glycerin | 5.000 |
| Sequestration agent | 0.100 |
| Teasel extract | 2.000 |
| Buddleia extract | 1.000 |
| Rosemary extract | 1.000 |
| Kaki extract | 3.000 |
| Pueraria extract | 3.000 |
| Pequi oil | 2.000 |
| Perfume | 0.500 |
| Preservatives | 1.000 |
| Purified water | QSP 100 |

Gel

| | |
|---|---|
| Butylene glycol | 2.000 |
| Neutralized thickening agent | 1.000 |
| Sequestration agent | 0.100 |
| Ethyl alcohol | 5.000 |
| Kaki extract | 3.000 |
| Pueraria extract | 3.000 |
| Teasel extract | 2.000 |
| Rosemary extract | 2.000 |
| Solubilizer | 1.000 |
| Perfume | 0.500 |
| Preservatives | 0.700 |
| Purified water | QSP 100 |

Serum

| | |
|---|---|
| Gelling agent | 0.600 |
| Glycerin | 2.000 |
| Butylene glycol | 3.000 |
| Sequestration agent | 0.100 |
| Rosemary extract | 1.000 |
| Teasel extract | 2.000 |
| Buddleia extract | 2.000 |
| Galangal extract | 1.000 |
| Kaki extract | 4.000 |
| Pueraria extract | 4.000 |
| Triglycerides | 2.000 |
| Silicon oils | 7.000 |
| Preservatives | 0.600 |
| Perfumes | 0.400 |
| Purified water | QSP 100 |

1. A cosmetic composition comprising:
an extract of Diospyros kaki,
an extract of Pueraria lobata, and
one or more formulation agents or additives compatible with cosmetic and dermatological use.

2. The cosmetic composition according to claim 1, wherein the extract of Diospyros kaki is an extract derived from calyxes of a Diospyros kaki plant and total polyphenol concentration is from about 2 to about 4 g/l.

3. The cosmetic composition according to claim 1, wherein the extract of Pueraria lobata is an extract derived from roots of a Pueraria lobata plant and total isoflavone content is from about 0.3 to about 0.6%.

4. The cosmetic composition according to claim 1, comprising about 0.1 to about 10% by weight of Diospyros kaki extract and about 0.1 to about 10% by weight of Pueraria lobata extract.

5. The cosmetic composition according to claim 1, comprising about 0.5 to about 5% by weight of Diospyros kaki extract and about 0.5 to about 5% by weight of Pueraria lobata extract.

6. The cosmetic composition according to claim 1, further comprising at least one active plant ingredient rich in flavonoids and/or in tannins selected from the group consisting of an extract of wild teasel (Dipsacus sylvestris), an extract of rosemary (Rosmarinus officinalis), an extract of galangal (Alpina officinarum) and an extract of buddleia (Buddleia davidii).

7. The cosmetic composition according to claim 1, wherein the one or more formulation agents or additives are selected from the group consisting of softeners, coloring agents, film-forming agents, surface-active agents, perfumes, preservatives, oils, glycols, vitamins and UV filters.

8. A method of preventing skin aging comprising applying a therapeutically effective amount of the composition of claim 1 to skin of a mammal.

9. The cosmetic composition according to claim 8, wherein the extract of Diospyros kaki is an extract derived from calyxes of a Diospyros kaki plant and total polyphenol concentration is from about 2 to about 4 g/l.

10. The cosmetic composition according to claim 8, wherein the extract of Pueraria lobata is an extract derived from roots of a Pueraria lobata plant and total isoflavone content is from about 0.3 to about 0.6%.

11. The cosmetic composition according to claim 8, comprising about 0.1 to about 10% by weight of Diospyros kaki extract and about 0.1 to about 10% by weight of Pueraria lobata extract.

12. The cosmetic composition according to claim 8, comprising about 0.5 to about 5% by weight of Diospyros kaki extract and about 0.5 to about 5% by weight of Pueraria lobata extract.

13. The cosmetic composition according to claim 8, further comprising at least one active plant ingredient rich in flavonoids and/or in tannins selected from the group consisting of an extract of wild teasel (Dipsacus sylvestris), an extract of rosemary (Rosmarinus officinalis), an extract of galangal (Alpina officinarum) and an extract of buddleia (Buddleia davidii).

14. The cosmetic composition according to claim 8, wherein the one or more formulation agents or additives are selected from the group consisting of softeners, coloring agents, film-forming agents, surface-active agents, perfumes, preservatives, oils, glycols, vitamins and UV filters.

15. A method of reducing skin aging comprising applying a therapeutically effective amount of the composition of claim 1 to skin of a mammal.

16. The cosmetic composition according to claim 15, wherein the extract of Diospyros kaki is an extract derived from calyxes of a Diospyros kaki plant and total polyphenol concentration is from about 2 to about 4 g/l.

17. The cosmetic composition according to claim 15, wherein the extract of Pueraria lobata is an extract derived from roots of a Pueraria lobata plant and total isoflavone content is from about 0.3 to about 0.6%.

18. The cosmetic composition according to claim 15, comprising about 0.1 to about 10% by weight of Diospyros kaki extract and about 0.1 to about 10% by weight of Pueraria lobata extract.

19. The cosmetic composition according to claim 15, comprising about 0.5 to about 5% by weight of Diospyros kaki extract and about 0.5 to about 5% by weight of Pueraria lobata extract.

20. The cosmetic composition according to claim 15, further comprising at least one active plant ingredient rich in flavonoids and/or in tannins selected from the group consisting of an extract of wild teasel (Dipsacus sylvestris), an extract of rosemary (Rosmarinus officinalis), an extract of galangal (Alpina officinarum) and an extract of buddleia (Buddleia davidii).

21. The cosmetic composition according to claim 15, wherein the one or more formulation agents or additives are selected from the group consisting of softeners, coloring agents, film-forming agents, surface-active agents, perfumes, preservatives, oils, glycols, vitamins and UV filters.
